(19) United States
(12) Patent Application Publication
(10) Pub. No.: US 2006/0228278 A1
(43) Pub. Date: Oct. 12, 2006

(54) Dry sterilizer

(76) Inventor: Takaharu Mizuno, Kanazawa-shi (JP)

Correspondence Address: FLYNN THIEL BOUTELL & TANIS, P.C., 2026 RAMBLING ROAD, KALAMAZOO, MI, 49008-1631, US

(21) Appl. No.: 11/101,230

(22) Filed: Apr. 7, 2005

(51) Int. Cl. A61L 2/04

(52) U.S. Cl. 422/307

(57) ABSTRACT

A conveying conveyer 2 which conveys vitreous vessels 1 such as vials or ampoules in a plurality of rows is disposed within a canister body 4. There are provided hot air supplying means 6 which blows a hot air against the vessels 1 on the conveying conveyer 2 to heat them to or above a predetermined temperature for purpose of sterilization, windows 34, 36 and 38 mounted in wall surfaces 4c, 4d and 18 of the canister body 4 for allowing infrared rays generated by the heated vessels 1 to transmit therethrough, and radiation pyrometers 40, 42 and 44 disposed externally of the canister body 4 for sensing infrared rays generated by the heated vessels 1 to measure the temperature of the vessels 1. Since the radiation pyrometers 40, 42 and 44 are located externally of the canister body, a confirmation of operation or a repair/replacement is facilitated, providing an excellent maintenance capability.

### BACKGROUND OF THE INVENTION AND RELATED ART STATEMENT

The present invention relates to a dry sterilizer which dries and sterilizes under heat medical vessels such as vials, ampoules, syringes and other vitreous vessels, and in particular, to a dry sterilizer with a thermometer which measures the temperature of vessels being heated.

In a dry sterilizer which dries and sterilizes vitreous medical vessels such as vials, ampoules or the like, the sterilization is achieved by blowing a hot air over vessels on a conveying conveyer which runs through a canister body to heat them. For a perfect sterilization of vessels, it is necessary to heat them to a predetermined temperature, and accordingly, the vessels are heated to the predetermined temperature by regulating the temperature and the wind speed of the hot air which is blown against vessels, the internal pressure, the running speed of the conveying conveyer and the like. During an actual operation, parameters such as the temperature and the wind speed of the hot air are measured and compared against experimental data which are previously determined to decide whether or not vessels are heated to the predetermined temperature. However, it is impossible to decide whether or not vessels have actually reached the predetermined temperature by merely controlling the parameters such as the temperature and the wind speed of the hot air. Accordingly, there is proposed a dry sterilizer which directly measures the temperature of vessels heated within the canister body (see Japanese Patent Publication No. 6-49057 (1994) or Japanese Laid-Open Patent Publication No. 5-132039 (1993), for example).

In the dry sterilizer disclosed in the first citation (Japanese Patent Publication No. 6-49057 (1994)) (the title of the invention in this citation reads a highly heated sterilization method), there are provided an inlet region, a drying/heating/sterilization region, a cooling region and an outlet region, and vessels to be sterilized pass through a radiation oven which defines the drying/heating/sterilizing region on a transport conveyer. A sterilization temperature of vitreous vessels in the radiation oven is continuously measured and monitored by a radiation pyrometer which is assembled into the sidewall of the radiation oven.

The dry sterilizer disclosed in the second citation (Japanese Laid-Open Patent Publication No. 5-132039 (1993)) (this citation refers to a thermally loadable package vessel as a sterilizing device) includes a tunnel-shaped oven through which vessels are guided on a conveying conveyer, with a thermal radiation measuring unit in a front wall of the oven and with mirrors disposed above an IR-radiator of the oven. The mirrors deflect heat radiation emitted upwardly from the vessels toward thermal radiation measuring unit, which inspects whether or not the vessels being conveyed are heated to a required sterilization temperature.

The dry sterilizers disclosed in the citations use the radiation pyrometer which is directly assembled into the wall surface of the oven. However, the radiation pyrometer and electric wires which are connected thereto have low heat resistant temperatures, and therefore, it is impossible to use them directly in the condition under which they are assembled. While such pyrometer may be assembled with heat insulating member interposed, but in order to check whether or not the radiation pyrometer is operating normally, there is a need to inspect on the order of once per year for confirming its operation. At this time, the radiation pyrometer must be dismounted and mounted. In an arrangement where the radiation pyrometer is mounted with the heat insulating member interposed, it takes time to dismount and mount it, resulting in a poor maintenance capability.

### OBJECT AND SUMMARY OF THE INVENTION

It is an object of the invention to solve above task by providing a dry sterilizer which is capable of accurately measuring the temperature of vessels which are dried and sterilized within a canister body while facilitating an inspection to ascertain the operation of radiation pyrometer and repair/replacement thereof, thus providing an excellent maintenance capability.

Above object is accomplished by providing a dry sterilizer including a conveyer disposed within a canister body for conveying vitreous vessels and hot air supply means for blowing a hot air to the vessels on the conveyer to heat them, the dry sterilizer further comprising a radiation pyrometer disposed outside the canister body and capable of detecting infrared rays in a particular wavelength range, and a window mounted on the canister body and formed of a material capable of transmitting those of infrared rays generated by the heated vessels which are in the wavelength range which is detected by the radiation pyrometer, thus allowing the radiation pyrometer to measure the temperature of the vessels.

In accordance with the invention, a radiation pyrometer capable of detecting infrared rays in a particular wavelength range is disposed on the outside of the canister body of the dry sterilizer, and a window formed of a material capable of transmitting those of infrared rays generated by the heated vessels which are in the wavelength range which is detected by the radiation pyrometer is mounted on the canister body, thereby allowing the temperature of the vessels to be accurately measured and facilitating a maintenance.

### BRIEF DESCRIPTION OF THE DRAWINGS

FIG. 1 is a longitudinal section showing an overall arrangement of a dry sterilizer according to one embodiment of the present invention;

FIG. 2 is a cross section of a heating region of the dry sterilizer; and

FIG. 3 is a cross section, to a enlarged scale, of an essential part shown in FIG. 2.

### DETAILED DESCRIPTION OF PREFERRED EMBODIMENT

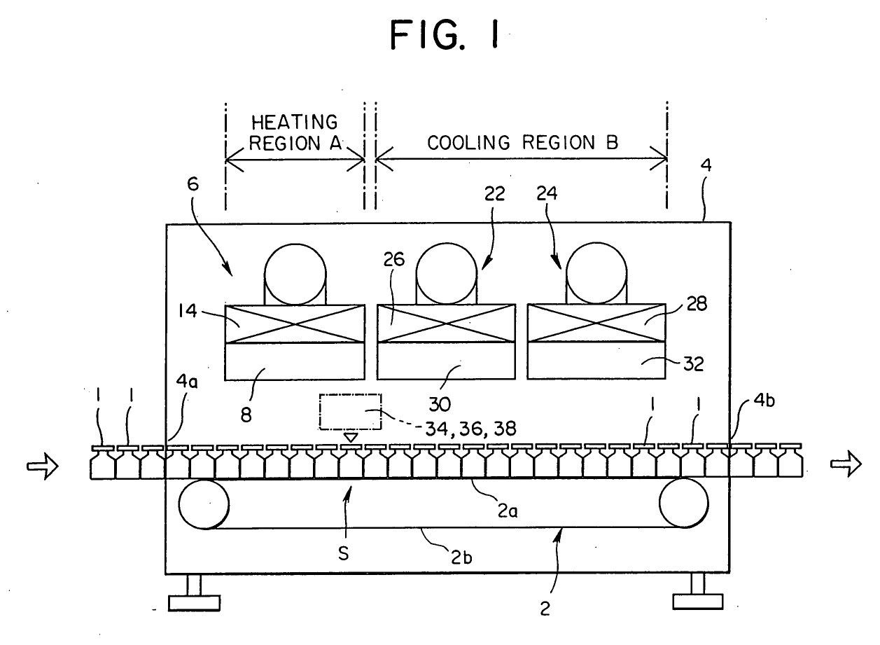

An embodiment of the present invention shown in the drawings will be described below. Dry sterilizer according to this embodiment includes a conveying conveyer 2 which continuously conveys a number of vessels 1 disposed in a plurality of rows in which vessels are in close contact with each other fore and after and right and left. Vessels 1 are carried through an opening (vessel inlet) 4a formed at one end (left end as viewed in FIG. 1) of a canister body 4, are heated and sterilized in a heating region A as they are conveyed through the canister body 4, cooled in a cooling region B, and discharged externally through an opening (vessel outlet) 4a formed at the other end (right end as viewed In FIG. 1) of the canister body 4 to be fed to a succeeding step.

The heating region A is disposed upstream and the cooling region B is disposed downstream within the canister body 4. A hot air supply means 6 is disposed in the heating region A to blow a hot air against vessels 1 which are conveyed by the conveyer 2 to heat them. The hot air supply means 6 comprises a hot air blowout opening 8 disposed above the conveyer 2 and facing downward, a heater 10 for heating the air which is to be blown out through the hot air blowout opening 8, and a blower 12 disposed between the hot air blowout opening 8 and the heater 10 to circulate the hot air. A HEPA filter 14 is mounted within the hot air blowout opening 8 to purify the hot air which is blown against the vessels 1 on the conveyer 2.

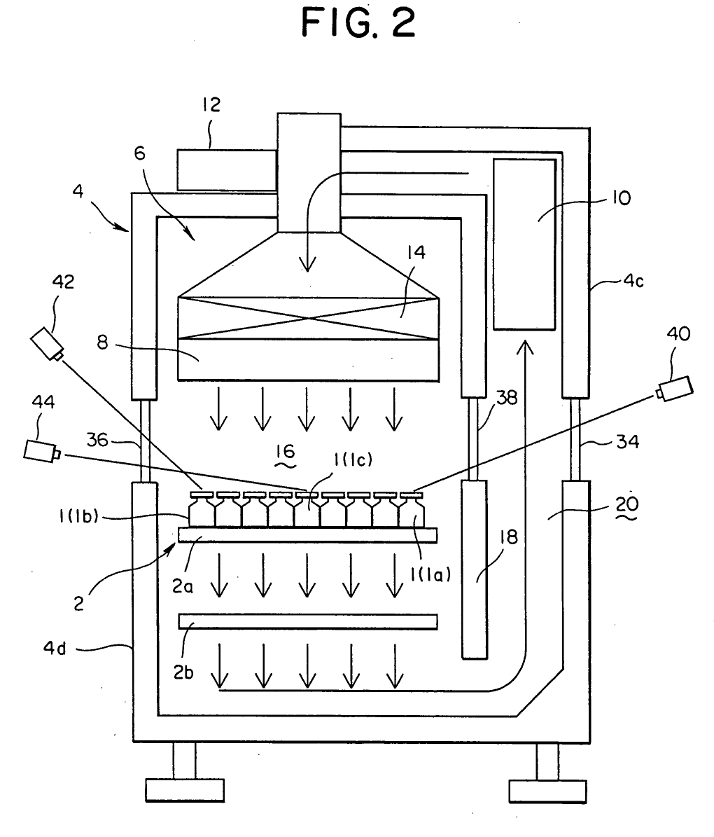

Toward one sidewall 4c (or right wall as viewed in FIG. 2), the canister body 4 is formed with a return flow path 20 which is partitioned by an internal septum 18 from a central space 16 in which the conveyer 2 is disposed, and the hot air which is blown out from the hot air blowout opening 8 downward into the central space 16 is blown against vessels 1 on the conveyer 2 to heat them, and then passes through the upper run 2a and through the lower run 2b of the conveyer to reach the bottom of the central space 16, and then flows below the lower end of the internal septum 18 to enter the return flow path 20 to be heated by the heater 10 again to be fed to the blowout opening 8.

Two cold air supply means 22 and 24 are disposed in the cooling region B which is located downstream of the heating region A. The cold supply means 22 and 24 include cold air blowout openings 30 and 32 in which HEPA filters 26 and 28 are mounted in the similar manner as the blowout opening 8 of the hot air supply means 6 disposed in the heating region A, an air feed blower which feeds air to the blowout openings 30 and 32 and an exhaust blower which exhausts air from within the canister body 4. Thus an external air is taken into the inside and a heated air is exhausted externally. In this embodiment, two cold air supply means 22 and 24 are provided to blow a cold air against the vessels 1 to cool them over an interval which is substantially twice the heating region A.

Windows 34, 36 and 38 formed of a fluoride crystal which is a material capable of transmitting infrared rays in a particular wavelength region are disposed in the opposite sidewalls 4c and 4d of the canister body 4 and in the internal septum 18. These windows 34, 36 and 38 are disposed adjacent to the downstream end of the heating region A (or a location indicated by character S in FIG. 1) and are at an elevation which is slightly above the upper surface of the vessels 1 which are being conveyed by the conveyer 2.

Radiation pyrometers 40, 42 and 44 are disposed on the outside of the windows 34 and 36 which are formed in the opposite sidewalls 4c and 4d of the canister body 4. These radiation pyrometers 40, 42 and 44 are designed to measure infrared rays in a particular wavelength range. By detecting those of infrared rays generated by materials which are in a wavelength region which can be measured, and by converting the intensity of the rays detected into the temperature, the temperature of the materials is measured. In the present embodiment, a wavelength range which is measured by the radiation pyrometers 40, 42 and 44 is set up to be 4.8-5.2 μm. However, it should be understood that the wavelengths measured by the radiation pyrometers are suitably set up depending on the type of the radiation pyrometers, the properties of materials, the temperature being measured and the situation under which the materials are placed, and are not limited to the values mentioned above.

The three radiation pyrometers 40, 42 and 44 are disposed so as to measure the top surface of the vessels 1 which are located at the downstream end S of the heating region A. In addition, the vessels 1 are disposed in 9 rows when they are being conveyed, and the radiation pyrometers are oriented toward outermost rows and the center row so that the temperature of vessels 1 which are disposed in these rows (1a, 1b and 1c) can be measured. In the present embodiment, the hot air is caused to circulate through the return flow path 2 which is disposed toward one sidewall 4c, and accordingly, the flow rate of the hot air varies crosswise of the canister body 4, causing a some temperature difference depending on the crosswise position. This is the reason of measuring the temperature at three locations for the opposite ends and the center of the vessels which are conveyed in nine rows. However, it is to be understood there is no need to measure the temperature of the vessels 1 (1a, 1b and 1c) at three locations, but the locations where the vessels 1 are detected and the number of such locations can be suitably chosen depending on the number of rows in which the vessels 1 are conveyed, the capacity of the hot air supply means 6, and the manner of circulation of the hot air within the canister body 4. In addition, the point of the vessel 1 which is subject to such measurement is not limited to the top surface thereof. For example, it is also possible to convey the vessels 1 by the conveyer 2 while maintaining the vessels spaced apart fore and aft, and the temperature adjacent to the bottom surface of the vessel 1 may be measured.

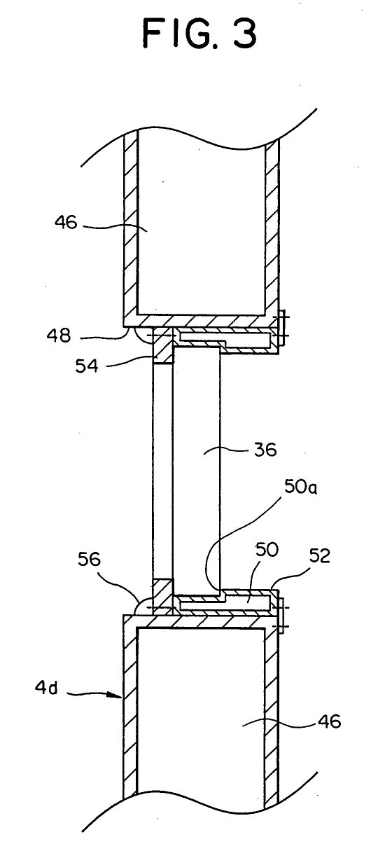

A structure for mounting the windows 34, 36 and 38 will be described with reference to FIG. 3. In the present embodiment, the windows 34, 36 and 38 are mounted on the wall surface at three locations (left and right sidewalls 4c and 4d and the internal septum 18) as shown in FIG. 2. Since the mounting structure is identical for each location, only one location (the window 36 in the left sidewall 4d shown in FIG. 2) will be described. The sidewall 4d is fitted with a heat insulating material 46, and is formed with a rectangular mounting opening 48. A mounting member 52 having a heat insulating material 50 packed therein is secured around the inner periphery of the mounting opening 48. The mounting member 52 has a greater thickness at the portion spaced from the sidewall 4d (or right-hand side as viewed in FIG. 3) while its outer portion is thinner, forming a step 50a therebetween. The window 36 which is formed of a fluoride crystal is fitted into the outer, thinner portion (left-hand portion as viewed in FIG. 3) of the mounting member 52. A mounting frame 54 is fitted to the outside of the window 36, and is secured to the mounting member 52. It will be noted that the window 36 which is formed of the fluoride crystal is held sandwiched between the inner side of the mounting frame 54 and the step 50a in the mounting member 52 to be secured in place. A seal member 56 is mounted between the outer surface (left side as viewed in FIG. 3) of the mounting frame 54 and the wall surface 4d to maintain a hermetic seal for the interior of the canister body 4.

In the present embodiment, calcium fluoride CaF2 is used for the material which forms the windows 34, 36 and 38. A window formed of calcium fluoride transmits infrared rays having wavelengths substantially equal to 0.13-8 μm. When measuring the temperature of the vitreous vessel 1 such as a vial or a ampoule with a radiation pyrometer, it is common that a wavelength to be measured is suitably 4.8 μm or greater. However, a normal glass absorbs infrared rays of wavelengths equal to or greater then 4.5 μm, and hence, if the normal glass is used for the windows 34, 36 and 38, the infrared rays can not reach the radiation pyrometer. For this reason, the windows 34, 36 and 38 used in the present embodiment are formed of calcium fluoride material. However, it is to be noted that the material for the windows 34, 36 and 38 is not limited to calcium fluoride, but that barium fluoride BaF2 or the like may also be used. A window formed of barium fluoride transmits infrared rays having wavelengths which are generally in a range of 0.18-12 μm, and thus is suitable for measuring the temperature of the vitreous vessels 1. Any material other than the fluoride material may be used for the windows 34, 36 and 38 provided it transmits infrared rays having wavelengths equal to or greater than 2.5 μm.

The vitreous vessels 1 such as vials or ampoules which are sterilized by the dry sterilizer are heated until the temperature measured by the radiation pyrometers 40, 42 and 44 becomes equal to or higher than 300° C. In the event the vessels 1 were detected, the temperature of which as measured by the radiation pyrometers 40, 42 and 44 is below 300° C., an operator stops the operation of the conveyer 2 and prevents such detected vessels 1 from being delivered out of the heating region A, and checks the parameters such as the temperature or the wind speed of the hot air to provide a judgment for a further operation. A control may be chosen such that the operation of the conveyer 2 is interrupted until it is confirmed that the temperature of the vessels 1 has reached 300° C.

The operation of the dry sterilizer constructed in the manner mentioned above will be described below. The vessels 1 which have been conveyed by conveying means which is external of the canister body 4 are carried into the canister body 4 through a vessel inlet 4a of the canister body 4 and placed on the conveyer 2 to be conveyed. In the present embodiment, the vessels 1 are arrayed in nine rows and are conveyed while they are contacting each other fore and aft and right and left.

The hot air supply means 6 is disposed at an upstream location within the canister body 4. The hot air supply means 6 delivers air which is heated by the heater 10 to HEPA filter 14 by the action of the blower 12 to be purified, and then blows the air from the hot air blowout opening 8 against the vessels 1 on the conveying conveyer 2. The hot air blown from the hot air blowout opening 8 is blown against the vessels 1 and then passes through the upper ran 2a, located toward the vessels, and the lower or return run 2b of the conveyer 2, to be diverted below the internal septum 18 into the return flow path 20, where it is heated by the heater 10 to be subsequently blown from the hot air blowout opening 8 for circulation. The vessels 1 are gradually heated as they are blown with the hot air while being conveyed by the conveyer 2.

When the vessels reach the downstream end (the location indicated by character S in FIG. 1) of the heating region A, their temperature is measured by the radiation pyrometer 40, 42 and 44 which are oriented toward the vessels 1 (1a, 1b and 1c) which are located at the opposite ends and at the center on the conveyer 2. Infrared rays which are radiated from the heated vessels 1 (1a, 1b and 1c) transmit through the windows 38, 34 and 36 which are formed of calcium fluoride to be detected by infrared ray sensors in the radiation pyrometers 40, 42 and 44. Infrared rays which are radiated from the vitreous vessels 1 such as vials or ampoules for transmit through the windows 34, 36 and 38 which are formed of calcium fluoride, permitting the transmission of wavelengths substantially in a range of 0.13-8 μm, and the temperature is measured by the radiation pyrometers 40, 42 and 44 which are designed to detect wavelengths in a range of 4.8-5.2 μm. For a normal glass window, a transmittance is high for short wavelengths equal or less than 2 μm, but the transmittance is reduced for a higher range of wavelengths, and in particular, there is substantially no transmission for a range of wavelengths equal to or greater than 5 μm, disabling the measurement with the radiation pyrometers 40, 42 and 44 which are set up as mentioned above. However, with the apparatus of the present embodiment, the use of the windows 34, 36 and 38 which are formed of calcium fluoride enables an accurate measurement of the temperature of the heated vessels 1. Since the radiation pyrometer 40, 42 and 44 are independently installed externally of the canister body 2, an inspection which confirms the operation is facilitated, providing an excellent maintenance capability.

In the present embodiment, when the temperature of the vessels as measured by the radiation pyrometers 40, 42 and 44 exceeds 300° C., a judgment is rendered that a required sterilization has been achieved, and the conveyer 2 is allowed to continue conveying the vessels 1 into the cooling region B for purpose of cooling. Two cold air supply means 22 and 24 are provided in the cooling region B, and a cold air is blown from the cold air blowout openings 30 and 32 against the vessels 1 to cool them to a predetermined temperature. The cooled vessels 1 are delivered externally through the vessel outlet 4a of the canister body 4 to be fed to a succeeding step.

In the event the temperature measured for the vessels 1 does not reach 300° C., the operation of the conveyer 2 is stopped, and parameters such as the temperature and the wind speed of the hot air are confirmed. In this instance, a control may be exercised such that the operation of the conveyer 2 is interrupted until it is confirmed that the temperature of the vessels 1 has exceeded 300° C. Alternatively, the temperature of the hot air fed from the hot air supply means 6 may be raised. The dry sterilizer of the present embodiment allows the temperature of the vessels 1 which are dried and sterilized within the canister body 4 to be accurately measured while facilitating an inspection to confirm the operation of the radiation pyrometers 40, 42 and 44, and repair/replacement thereof to provide an excellent maintenance capability.

In the dry sterilizer constructed in the manner mentioned above, parameters such as the temperature and the wind speed of the hot air, the internal pressure or the like are detected at a given time interval, and such data is recorded. The radiation pyrometers 40, 42 and 44 detect the temperature at a given interval, and such temperature is also recorded. It is possible to utilize such recorded data effectively in order to guarantee the quality of production.

In the embodiment described above, the radiation pyrometer 40 which is installed on the side of the return flow path 20 measures the temperature of the vessels 1 through the pair of windows 34 and 38 while the radiation pyrometers 42 and 44 installed on the other side (left side as viewed in FIG. 2) measure the temperature of the vessels through the single window 36. The number of windows is not limited to any particular value, and a single window or a plurality windows may be used. Although the transmittance of infrared rays varies depending on the numbers of windows which they transmit, the temperature can be determined accurately by applying a corrective calculation in association with the radiation pyrometers 40, 42 and 44. In the described embodiment, the three radiation pyrometers 40, 42 and 44 are secured at fixed positions to measure the temperature of vessel 1 (1a, 1b and 1c) which are located at the same position, but the radiation pyrometers 40, 42 and 44 may be connected with drive means to move them in the direction in which the vessels are conveyed or in a crosswise direction or in both directions.

1. A dry sterilizer including a conveying conveyer disposed within a canister body for conveying vitreous vessels, and hot air supply means for blowing hot air against vessels on the conveying conveyer to heat them; further comprising
a radiation pyrometer disposed externally of the canister body and capable of detecting infrared rays in a particular range of wavelengths;
and a window formed of a material which is capable of transmitting those of infrared rays generated by the heated vessels which are in a range of wavelengths which are detected by the radiation pyrometer, the window being mounted on the canister body to allow the temperature of the vessels to be measured with the radiation pyrometer.

2. A dry sterilizer according to claim 1 in which the vessels are medical vessels such as vials, ampoules, syringes or the like.

3. A dry sterilizer according to claim 1 in which the material which is used to form the window comprises fluoride crystal.

4. A dry sterilizer according to claim 3 in which the material which forms the window comprises barium fluoride or calcium fluoride.

5. A dry sterilizer according to claim 1, further comprising a heating region in which the vessels are heated for a given interval, the radiation pyrometer measuring the temperature of vessels as they are located at a downstream end of the heating region.

6. A dry sterilizer according to claim 1 in which the vessels are conveyed by the conveyer in a plurality of rows and there are a plurality of said radiation pyrometers to measure the temperature of a plurality of vessels.
